(19) United States
(12) Patent Application Publication
(10) Pub. No.: US 2010/0172850 A1
(43) Pub. Date: Jul. 8, 2010

(54) WATER-IN-OIL TYPE SUNSCREEN COSMETIC

(75) Inventor: Daisuke MITSUI, Nakatsugawa-shi (JP)

Correspondence Address: YI LI, CUSPA TECHNOLOGY LAW ASSOCIATES, 11820 SW 107 AVENUE, MIAMI, FL, 33176, US

(73) Assignee: TOKIWA CORPORATION, Nakatsugawa-shi (JP)

(21) Appl. No.: 12/649,870

(22) Filed: Dec. 30, 2009

(30) Foreign Application Priority Data
Jan. 8, 2009 (JP) JP2009-002534

(51) Int. Cl. A61K 8/27; A61Q 17/04; A61K 8/29

(52) U.S. Cl. 424/59

(57) ABSTRACT

The present invention provides a water-in-oil type sunscreen cosmetic with superior emulsion stability, which hardly whiten the skin when applied, is easy to spread, barely oily, and have excellent applicability. The water-in-oil type sunscreen cosmetic comprises particulate zinc oxide subjected to surface hydrophobizing treatment and/or particulate titanium dioxide subjected to surface hydrophobizing treatment, silicone branched type polyglycerin-modified silicone and isostearic acid, wherein the content of the silicone branched type polyglycerin-modified silicone is 1.5-10% by mass of the total amount of the cosmetic, and the content of the isostearic acid is 0.1-10% by mass of the total amount of the cosmetic.

### TECHNICAL FIELD

The present invention relates to a water-in-oil type sunscreen cosmetic.

### BACKGROUND

Due to the water resistance to resist sweat and sea water required by sunscreen cosmetics, water-in-oil type cosmetics are the mainstream of sunscreen cosmetics. Organic-based UV absorbents such as cinnamic acid-based compounds and benzophenone-based compounds, and inorganic-based UV scattering agents such as particulate titanium dioxide and particulate zinc oxide, as components to avoid skin damage by UV are added in sunscreen cosmetics.

Recently, cosmetics milder to skin have always been sought for, and therefore attention is focused on nonchemical type sunscreen cosmetics without addition of organic-based UV absorbents. Such sunscreen cosmetics achieved desired UV screening effect by increasing the addition amount of the inorganic-based UV scattering agents mentioned above.

It is able to both exhibit transparency and effectively screen UV by finely dispersing particles such as particulate titanium dioxide and particulate zinc oxide into cosmetics. However, when particles are not thoroughly dispersed and are agglomerated therebetween, problems of whitening on the skin applied with cosmetic and lowered UV screening effect will emerge.

Various researches have been done on inhibition of the agglomeration of powders in water-in-oil type cosmetics. For instance, patent reference 1 below has proposed a powder composition comprising silicone branched type polyglycerin-modified silicone.

### PRIOR ART DOCUMENT

Patent reference 1: Japanese patent laid-open publication No. 2004-169015.

### SUMMARY OF THE INVENTION

### Problems to be Solved

The above-mentioned silicone branched type polyglycerin-modified silicone is effective for inhibiting agglomeration of powders. However, for water-in-oil type sunscreen cosmetics, decrease in emulsion stability when the particles are finely dispersed needs to be suppressed in addition to prevention of particulates agglomeration. It is shown in the research of the inventor that it is hard to maintain practically satisfactory emulsion stability if silicone branched type polyglycerin-modified silicone alone is added. Furthermore, in view of water resistance and oiliness suppression, nonpolar oils such as silicone oil are often added in water-in-oil type sunscreen cosmetics, although it is even more difficult to obtain sufficient emulsion stability because the interface strength decreases in such systems. Thus, even the powder composition in above patent reference 1 needs to be further improved on emulsion stability.

Further, techniques of adding organic modified clay minerals and polyglycerin-modified silicone are known as methods to stabilize emulsion in water-in-oil emulsified material. However, since such methods make it difficult for cosmetics to spread, they can not be deemed as effective methods regarding applicability of the cosmetics.

In the light of the above situation, the present invention is accomplished and the objective thereof is to provide a water-in-oil type sunscreen cosmetic with superior emulsion stability which hardly whiten the skin when applied, is easy to spread, barely oily, and have excellent applicability.

### Solutions for Solving the Problems

To solve the above problems, the water-in-oil type sunscreen cosmetic of the present invention is characterized in that: comprising particulate zinc oxide subjected to surface hydrophobizing treatment and/or particulate titanium dioxide subjected to surface hydrophobizing treatment; as well as silicone branched type polyglycerin-modified silicone and isostearic acid, the content of the silicone branched type polyglycerin-modified silicone being 1.5-10% by mass on the basis of the total amount of the cosmetic, and the content of the isostearic acid being 0.1-10% by mass on the basis of the total amount of the cosmetic.

Having the composition described above, the water-in-oil type sunscreen cosmetic of the present invention achieves UV screening effect imparted by above-mentioned particulates while showing superior emulsion stability, hardly whiten the skin when applied, and achieving excellent applicability such as ease to spread and little oiliness.

Further, it is difficult to sufficiently avoid the problem of whitening on the skin when applied on skin when the content of the silicone branched type polyglycerin-modified silicone is less than 1.5% by mass on the basis of the total amount of the cosmetic, while the cosmetic becomes readily oily when the content exceeds 10% by mass. Additionally, it is difficult to sufficiently ensure the emulsion stability of the cosmetic when the content of the isostearic acid is less than 0.1% by mass on the basis of the total amount of the cosmetic, while the cosmetic becomes hard to spread and readily oily when the content is more than 10% by mass.

Preferably, for the water-in-oil type sunscreen cosmetic of the present invention, the surface of the particulate zinc oxide and particulate titanium dioxide mentioned above is subjected to hydrophobizing treatment with silicone oil or metallic soap in view of transparency and UV screening effect.

Further, the average primary particle size of the particulate zinc oxide and particulate titanium dioxide mentioned above are preferred to be 1-50 nm in view of transparency and UV screening effect.

Furthermore, for the water-in-oil type sunscreen cosmetic of the present invention, the HLB value of the above silicone branched type polyglycerin-modified silicone is preferred to be 2-5. Even more superior emulsion stability could be achieved by adding the silicone branched type polyglycerin-modified silicone.

In addition, the water-in-oil type sunscreen cosmetic of the present invention can be formulated into sunscreen cosmetics without organic UV absorbent. In this case, water-in-oil type sunscreen cosmetics with superior emulsion stability and applicability which are milder to skin may be effectively achieved.

### EFFECTS OF THE INVENTION

According to the present invention, a water-in-oil type sunscreen cosmetic with superior emulsion stability which hardly whiten the skin when applied, is easy to spread, barely oily, and have excellent applicability can be provided.

### BEST MODES FOR CARRYING OUT THE INVENTION

Next, the water-in-oil type sunscreen cosmetic of the present invention is described in details.

The particulate zinc oxide and particulate titanium dioxide used in the present invention refer to substances having an average primary particle size of 100 nm or less. In the present invention, substances using the particulates as base material with the surface thereof hydrophobically treated are used. As methods for hydrophobizing treatment, silicone treatment, metallic soap treatment, fluorine treatment, amino acid treatment, fatty acid treatment, alkyl phosphate treatment and so on can be mentioned. Among them, substances treated with silicone or metallic soap are preferred. As silicone used in silicone treatment, methyl polysiloxane, methylphenylpolysiloxane, methylhydrogenpolysiloxane, methylcyclicpolysiloxane, dimethylsiloxane•methyl (polyoxypropylene)siloxane polymer and so on can be mentioned. As metallic soap used in metallic soap treatment, fatty acid metallic salts such as aluminum stearate and zinc stearate can be mentioned.

Further, the average primary particle size of the particulate zinc oxide and particulate titanium dioxide is preferably 1-100 nm, more preferably 1-50 nm in view of transparency and UV screening effect.

As particulate zinc oxide with the above-mentioned average primary particle size, commercially available substances such as “MZ-303S” (manufactured by Tayca Corporation, trade name, treated with methicone, average primary particle size 30-40 nm), “MZ-303M” (manufactured by Tayca Corporation, trade name, treated with dimethicone, average primary particle size 30-40 nm), and “FINEX-50S-LP2” (manufactured by Sakai Chemical Co., trade name, treated with (dimethicone/methicone) copolymer, average primary particle size 20 nm) can be mentioned.

Additionally, as particulate titanium dioxide with the above-mentioned average primary particle size, commercially available substances such as “TTO-V-4” (manufactured by Ishihara sangyo Kaisha, trade name, treated with aluminum hydroxide and stearic acid, average primary particle size 10 nm), “ST-455” (manufactured by Titan Kogyo, Ltd., trade name, treated with aluminum hydroxide and stearic acid, average primary particle size 20 nm), and “TTO-55(S)” (manufactured by Ishihara sangyo Kaisha, trade name, treated with aluminum hydroxide and organic siloxane, average primary particle size 40 nm) can be mentioned.

In the case that only one of the above particulate zinc oxide and the above particulate titanium dioxide is added in the cosmetic of the present invention, the content of the above particulate zinc oxide is preferably 7%-25% by mass on the basis of the total amount of the cosmetic, while the content of the above particulate titanium dioxide is preferably 5%-20% by mass on the basis of the total amount of the cosmetic.

In addition, it is more preferable that both the above particulate zinc oxide and the above particulate titanium dioxide are added in the cosmetic of the present invention, and the total amount of both particulates is preferably 10%-30% by mass on the basis of the total amount of the cosmetic. Preferably, the mixing ratio between the above particulate zinc oxide and the above particulate titanium dioxide is 1:1-4:1 in mass ratio.

As the silicone branched type polyglycerin-modified silicone used in the present invention, for examples, silicone branched type polyglycerin-modified silicones such as silicone branched type polyglycerin-modified silicone described in Japanese patent laid-open publication No. 2004-169015, commercial “KF-6100” (manufactured by Shin-Etsu Chemical Co., trade name, cosmetic presenting name: polyglyceryl-3 disiloxane dimethicone), “KF-6104” (manufactured by Shin-Etsu Chemical Co., trade name, cosmetic presenting name: polyglyceryl-3 polydimethylsiloxy ethyl dimethicone), and “KF-6105” (manufactured by Shin-Etsu Chemical Co., trade name, cosmetic presenting name: lauryl polyglyceryl-3 polydimethylsiloxy ethyl dimethicone) can be mentioned.

It is noteworthy that the silicone branched type polyglycerin-modified silicone described in Japanese patent laid-open publication No. 2004-169015 is the silicone branched type polyglycerin-modified silicone represented by general formula (1) below.

R¹ₐR²_{b}R³_{c}SiO_{(4-a-b-c)/2} (1)

[In the formula, R¹ is same or different organic group(s) selected from alkyl having 1-30 carbon atoms, aryl, aralkyl, fluoro-substituted alkyl, amino-substituted alkyl, carboxyl-substituted alkyl, or an organic group represented by general formula (2) below,

—C_{d}H_{2d}—O—(C₂H₄O)ₑ(C₃H₆O)_{f}R⁴ (2)

R² is a polyglycerin derivative represented by general formulae (3) and/or (4) below,

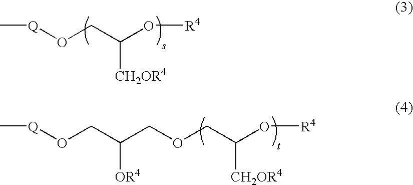

(in the formulae, R⁴ is hydrogen, alkyl with 1-30 carbon atoms, or an organic group represented by R⁵—(CO)— in which R⁵ is a hydrocarbon group with 1-30 carbon atoms. Q is a divalent hydrocarbon group with 3-20 carbon atoms that may comprise ether bond or ester bond otherwise; s is an integer of 2-20; and t is an integer of 1-20.) R³ is a silicon-containing group represented by general formula (5) below.

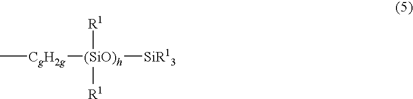

(In the formula, a, b, c meet 1.0≦a≦2.5, 0.001≦b≦1.5, 0.001≦c≦1.5, respectively; d, e, f are integers that meet 0≦d≦15, 0≦e≦50, 0≦f≦50 respectively, g is an integer that meets 1≦g≦5, and h is an integer that meets 0≦h≦500.)]

In order to achieve more superior emulsion stability, the HLB value of the silicone branched type polyglycerin-modified silicone is preferred to be 2-5.

The content of the silicone branched type polyglycerin-modified silicone in the water-in-oil type sunscreen cosmetic of the present invention is 1.5%-10% by mass on the basis of the total amount of the cosmetic, but preferably 2.5%-5% by mass in view of reduction of whitening and oiliness.

The water-in-oil type sunscreen cosmetic of the present invention comprises 0.1%-10% by mass of isostearic acid on the basis of the total amount of the cosmetic. From the perspective of emulsion stability and oiliness reduction, the content of the isostearic acid is preferably 0.2%-5% by mass.

The water-in-oil type sunscreen cosmetic of the present invention may comprise oil agents as components in addition to above essential components. As such oil agent, for example, hydrocarbons such as silicone oil, paraffin wax, ceresine wax, microcrystalline wax, Fischer-Tropsch wax, polyethylene wax, liquid paraffin, squalane, vaseline, polyisobutylene, and polybutene; natural waxes such as carnauba wax, bee wax, and candelilla wax; esters such as octyl methoxy cinnamate, glyceryl tribehenate, jojoba oil, cetyl isooctanoate, isopropyl myristate, glyceryl trioctanoate, diglyceryl triisostearate, dipentaerythritol fatty acid ester, and phytosterol Macadamia nut oil fatty acid ester; fatty acids such as stearic acid, behenic acid, and 12-hydroxystrearic acid; higher alcohols such as cetanol, stearyl alcohol, and behenyl alcohol; greases such as olive oil, castor oil, and haze wax; lanolin derivatives such as isopropyl lanolin fatty acid ester, and lanolin alcohol; fluorine-based oil agents such as perfluoropolyether, perfluorodecane, and perfluorooctane; and so on are mentioned. These can be used alone or in combination of two or more.

As the silicone oil mentioned above, for example, dimethylpolysiloxane, methylphenylpolysiloxane, decamethyl cyclopentasiloxane, octamethyl cyclotetrasiloxane, tri(trimethylsiloxy)methylsilane, tri(trimethylsiloxy)alkylsilane, trimethylsiloxy silicic acid, methylphenylpolysiloxane with high polymerization degree, partial-crosslinked organic polysiloxane, polyoxy-modified organic polysiloxane, crosslinked polyether-modified methylpolysiloxane, methacrylic-modified polysiloxane, stearyl-modified methylpolysiloxane, oleyl-modified methylpolysiloxane, behenyl-modified methylpolysiloxane, polyvinyl pyrrolidone-modified methylpolysiloxane, dimethylpolysiloxane with high polymerization degree, polyoxyalkylene•alkylmethylpolysiloxane•methylpolysiloxane copolymer, alkoxy-modified polysiloxane, and fluoro-modified polysiloxane and so on can be mentioned.

On the basis of the total amount of the cosmetic, the content of the oil agent in the water-in-oil type sunscreen cosmetic of the present invention is preferably 20%-60% by mass, more preferably 30%-60% by mass.

In view of water resistance and oiliness suppression, preferably the water-in-oil type sunscreen cosmetic of the present invention comprises silicone oil. In this case, the content of the silicone oil is preferably 25%-75% by mass, more preferably 30%-50% by mass on the basis of the total amount of the cosmetic. According to the present invention, even if the water-in-oil type sunscreen cosmetic comprises silicone oil in an amount within the above range, it can obtain the water-in-oil type sunscreen cosmetic having the following properties: it shows superior emulsion stability, is unlikely to whiten the skin when applied, easy to spread and even less oily, and have more superior applicability while having the UV screening effect imparted by the particulates of the present invention as described above.

Within the scope in which the effect of the present invention is not impaired, any powder components in addition to the particulate zinc oxide above and the particulate titanium dioxide above can be adequately added in the water-in-oil type sunscreen cosmetic of the present invention. Such powder component is not particularly limited, provided that it is commonly used in cosmetics, and the examples thereof include inorganic extender pigments such as mica, synthetic mica, sericite, mica titanium, iron oxide red-coated mica titanium, boron nitride, bismuth oxychloride, tabular alumina, talc, kaolin, magnesium carbonate, calcium carbonate, aluminum silicate, magnesium silicate, aluminum magnesium silicate, calcium silicate, and silicic acid anhydride; inorganic white pigments such as titanium dioxide and cerium oxide; inorganic coloring pigments such as iron oxide red, yellow iron oxide, black iron oxide, ultramarine, Prussian blue, and carbon black; organic coloring pigments such as tar-based colorants, and natural colorants; organic powders such as nylon powder, silicone resin powder, silk powder, polystyrene, polyethylene powder, crystalline cellulose, and N-acyl lysine. Substances treated with fluorine compounds, silicone-based compounds, metallic soap, wax, grease, and hydrocarbon etc. on the surface or untreated substances can be used as such powder components. Further, these powder components can be used alone or in combination of two or more.

Aqueous components may be added in the water-in-oil type sunscreen cosmetic of the present invention. Such aqueous component is not particularly limited, provided that it is commonly used in cosmetics, and the examples thereof include lower alcohols such as ethanol and isopropanol; glycols such as propylene glycol, 1,3-butylene glycol, dipropylene glycol, and polyethylene glycol; glycerols such as glycerin, diglycerin, and polyglycerin; saccharides such as sorbitol, maltitol, sucrose, starch sugar, and lactitol; water soluble polymers such as guar gum, sodium chondroitin sulfate, sodium hyaluronate, arabic gum, sodium alginate, xanthan gum, carrageenan, methyl cellulose, hydroxyethyl cellulose, carboxymethyl cellulose, carboxyvinyl polymer, polyvinyl alcohol, polyvinylpyrrolidone, alkylated carboxyvinyl polymer, and sodium polyacrylate; acids such as citric acid, lactic acid, malic acid, and tartaric acid or salts thereof; and salts such as sodium chloride and magnesium chloride. These can be used alone or in combination of two or more.

In addition to the above optional components, components frequently added in cosmetics including emulsifier, spice, preservative, chelator, antioxidant, bactericidal agent, and cosmetic component etc. may be added in the water-in-oil type sunscreen cosmetic of the present invention according to purpose within the scope in which the effect of the present invention is not impaired.

Water content in the water-in-oil type sunscreen cosmetic of the present invention is preferably 5%-50% by mass, more preferably 15%-40% by mass on the basis of the total amount of the cosmetic.

The water-in-oil type sunscreen cosmetic of the present invention may be produced according to, e.g., the following orders. First, the particulate titanium dioxide involved in the present invention, the silicone branched type polyglycerin-modified silicone involved in the present invention and oil agent are evenly mixed to give mixture (A). On the other hand, the particulate zinc oxide, isostearic acid, oil agent and emulsifier involved in the present invention are evenly mixed to give mixture (B). The addition amount of the silicone branched type polyglycerin-modified silicone and isostearic acid involved in the present invention are set so that the amounts thereof in the final cosmetic obtained fall within the ranges mentioned above.

The above-mentioned mixing may be proceeded for example by using three-roll mill, ball mill, or bead mill.

Next, mixture (A) and mixture (B) are evenly mixed to yield an oil phase mixture. This mixing may be done for example by using disper or homomixer.

Aqueous phase mixture containing water and aqueous components are then added into the oil phase mixture while emulsification is carrying out, thereby giving the water-in-oil type sunscreen cosmetic. Emulsification may be accomplished by using homomixer or disper etc.

It is noteworthy that the preparation method for the water-in-oil type sunscreen cosmetic of the present invention is not limited to the method mentioned above.

The water-in-oil type sunscreen cosmetic of the present invention is a cosmetic that can provide UV protection function, for example to be used as sunscreen agent, foundation, cosmetic base and so on.

### EXAMPLES

The present invention is illustrated in more details by examples below, but not limited thereto.

### Examples 1-7 and Comparative Examples 1-10

Water-in-oil type sunscreen cosmetics with the composition shown in Tables 1-2 are prepared respectively according to the following preparation method. By test methods and determination criteria shown below, items like “emulsion stability”, “spreading”, “non-oiliness”, and “whitening” are evaluated and determined for the resulting water-in-oil type sunscreen cosmetics. The determination results for these items are shown in Tables 1-2. In addition, the unit for each component in the tables is mass % based on the total amount of the cosmetic unless otherwise stated.

TABLE 1

| Components | | Example 1 | Example 2 | Example 3 | Example 4 | Example 5 | Example 6 | Example 7 |
|---|---|---|---|---|---|---|---|---|
| 1 | Decamethyl cyclopentasiloxane | 33 | 33 | 33 | 35 | 26.5 | 32.4 | 23.5 |
| 2 | KF-96A-6cs (dimethicone) | 9 | 9 | 9 | 9 | 9 | 6 | 9 |
| 3 | Particulate titanium dioxide | 9 | 9 | 9 | 9 | 9 | 9 | 9 |
| 4 | Particulate zinc oxide | 18 | 18 | 18 | 18 | 18 | 18 | 18 |
| 5 | Diisostearyl malate | 2 | 2 | 2 | 2 | 2 | 6 | 2 |
| 6 | Silicone branched type polyglycerin- | 3.5 | — | — | — | — | — | — |
| | modified silicone*¹ | | | | | | | |
| 7 | Silicone branched type polyglycerin- | — | 3.5 | — | 1.5 | 10 | 3.5 | 3.5 |
| | modified silicone*² | | | | | | | |
| 8 | Silicone branched type alkyl•polyglycerin- | — | — | 3.5 | — | — | — | — |
| | modified silicone*³ | | | | | | | |
| 9 | Polyether-modified silicone*⁴ | — | — | — | — | — | — | — |
| 10 | Crosslinked type alkyl•polyglycerin- | — | — | — | — | — | — | — |
| | modified silicone*⁵ | | | | | | | |
| 11 | Isostearic acid | 0.5 | 0.5 | 0.5 | 0.5 | 0.5 | 0.1 | 10 |
| 12 | Stearic acid | — | — | — | — | — | — | — |
| 13 | Palmitic acid | — | — | — | — | — | — | — |
| 14 | Behenic acid | — | — | — | — | — | — | — |
| 15 | Water | 20 | 20 | 20 | 20 | 20 | 20 | 20 |
| 16 | 1,3-BG | 4 | 4 | 4 | 4 | 4 | 4 | 4 |
| 17 | Glycerin | 1 | 1 | 1 | 1 | 1 | 1 | 1 |
| Total | | 100 | 100 | 100 | 100 | 100 | 100 | 100 |
| Evaluation | Emulsion stability | ◯ | ⊚ | ⊚ | ⊚ | ⊚ | ◯ | ⊚ |
| items | Spreading | ⊚ | ⊚ | ⊚ | ⊚ | ⊚ | ◯ | ◯ |
| | Non-oiliness | ⊚ | ⊚ | ⊚ | ⊚ | ◯ | ⊚ | ◯ |
| | Whitening | ⊚ | ⊚ | ⊚ | ◯ | ⊚ | ⊚ | ⊚ |

TABLE 2

| | | | | | | | | | Com- | Com- | Com- |
|---|---|---|---|---|---|---|---|---|---|---|---|
| | | Com- | Com- | Com- | Com- | Com- | Com- | Com- | parative | parative | parative |
| | | parative | parative | parative | parative | parative | parative | parative | Exam- | Exam- | Example |
| Components | | Example 1 | Example 2 | Example 3 | Example 4 | Example 5 | Example 6 | Example 7 | ple 8 | ple 9 | 10 |
| 1 | Decamethyl | 35.5 | 24.5 | 33 | 29.5 | 32.45 | 21.5 | 33 | 33 | 33 | 33.5 |
| | cyclopentasiloxane | | | | | | | | | | |
| 2 | KF-96A-6cs | 9 | 9 | 9 | 9 | 6 | 9 | 9 | 9 | 9 | 9 |
| | (dimethicone) | | | | | | | | | | |
| 3 | Particulate titanium | 9 | 9 | 9 | 9 | 9 | 9 | 9 | 9 | 9 | 9 |
| | dioxide | | | | | | | | | | |
| 4 | Particulate zinc oxide | 18 | 18 | 18 | 18 | 18 | 18 | 18 | 18 | 18 | 18 |
| 5 | Diisostearyl malate | 2 | 2 | 2 | 2 | 6 | 2 | 2 | 2 | 2 | 2 |
| 6 | Silicone branched type | — | — | — | — | — | — | — | — | — | — |
| | polyglycerin-modified | | | | | | | | | | |
| | silicone*¹ | | | | | | | | | | |
| 7 | Silicone branched type | 1 | 12 | — | — | 3.5 | 3.5 | 3.5 | 3.5 | 3.5 | 3.5 |
| | polyglycerin-modified | | | | | | | | | | |
| | silicone*² | | | | | | | | | | |
| 8 | Silicone branched type | — | — | — | — | — | — | — | — | — | — |
| | alkyl•polyglycerin- | | | | | | | | | | |
| | modified silicone*³ | | | | | | | | | | |
| 9 | Polyether-modified | — | — | 3.5 | 2 | — | — | — | — | — | — |
| | silicone*⁴ | | | | | | | | | | |
| 10 | Crosslinked type | — | — | — | 5 | — | — | — | — | — | — |
| | alkyl•polyglycerin- | | | | | | | | | | |
| | modified silicone*⁵ | | | | | | | | | | |
| 11 | Isostearic acid | 0.5 | 0.5 | 0.5 | 0.5 | 0.05 | 12 | — | — | — | — |
| 12 | Stearic acid | — | — | — | — | — | — | 0.5 | — | — | — |
| 13 | Palmitic acid | — | — | — | — | — | — | — | 0.5 | — | — |
| 14 | Behenic acid | — | — | — | — | — | — | — | — | 0.5 | — |
| 15 | Water | 20 | 20 | 20 | 20 | 20 | 20 | 20 | 20 | 20 | 20 |
| 16 | 1,3-BG | 4 | 4 | 4 | 4 | 4 | 4 | 4 | 4 | 4 | 4 |
| 17 | Glycerin | 1 | 1 | 1 | 1 | 1 | 1 | 1 | 1 | 1 | 1 |
| Total | | 100 | 100 | 100 | 100 | 100 | 100 | 100 | 100 | 100 | 100 |
| Evaluation | Emulsion stability | ◯ | ⊚ | ⊚ | ⊚ | X | ⊚ | X | X | X | X |
| items | Spreading | ◯ | ◯ | ⊚ | ◯ | ⊚ | Δ | X | X | X | ⊚ |
| | Non-oiliness | ⊚ | Δ | ◯ | ⊚ | ⊚ | Δ | Δ | Δ | X | ⊚ |
| | Whitening | Δ | ⊚ | X | Δ | ⊚ | ⊚ | ◯ | ◯ | ◯ | ⊚ |

### <Preparation Method for Water-in-Oil Type Sunscreen Cosmetics>

(1) Components 2, 3, and 6-10 are evenly mixed according to the ratio shown in Tables 1-2 using three-roll mill to give mixture A.
(2) On the other hand, components 1, 4, 5, 11-14 (solid fatty acids are melt by heating) are evenly mixed according to the ratio shown in Tables 1-2 using homomixer to give mixture B.
(3) Mixture A and mixture B obtained above are evenly mixed by homomixer to yield oil phase mixture C.
(4) On the other hand, components 15-17 are mixed by homomixer to yield aqueous phase mixture D.
(5) The above resulting aqueous phase mixture D is added into the oil phase mixture C obtained above and mixed by homomixer so as to emulsify followed by degassing, and then filled in containers to give water-in-oil type sunscreen cosmetics.

Additionally, the following substances are used for the components shown in Tables 1-2.

Decamethyl cyclopentasiloxane: “TSF-405” (manufactured by Momentive Performance Materials Japan).
KF-96A-6cs (dimethicone): manufactured by Shin-Etsu Chemical Co., trade name.
Particulate titanium dioxide: “TTO-V-4” (manufactured by Ishihara sangyo Kaisha, trade name, treated with aluminum hydroxide and stearic acid, average primary particle size 10 nm).
Particulate zinc oxide: “FINEX-50S-LP2” (manufactured by Sakai Chemical Co., treated with (dimethicone/methicone) copolymer, average primary particle size 20 nm).
Diisostearyl malate: “COSMOL 222(s)” (manufactured by the Nisshin Oillio Group Co.).
Silicone branched type polyglycerin-modified silicone*¹: polyglyceryl-3 disiloxane dimethicone, trade name “KF6100” (manufactured by Shin-Etsu Chemical Co.) (HLB: 7).
Silicone branched type polyglycerin-modified silicone*²: polyglyceryl-3 polydimethylsiloxyethyl dimethicone, trade name “KF6104” (manufactured by Shin-Etsu Chemical Co.) (HLB: 4.5).
Silicone branched type alkyl•polyglycerin-modified silicone*³: lauryl polyglycerin-3 polydimethylsiloxyethyl dimethicone, trade name “KF6105” (manufactured by Shin-Etsu Chemical Co.) (HLB: 4.5).
Polyether-modified silicone*⁴: PEG10 dimethicone, trade name “KF6017” (manufactured by Shin-Etsu Chemical Co.).
Crosslinked type alkyl•polyglycerin-modified silicone*⁵: polyglyceryl-3/laurylpolydimethylsiloxyethyl dimethicone crosslinked polymer 30%+cyclopentasiloxane 70%, trade name “KSG850Z” (manufactured by Shin-Etsu Chemical Co.).
Isostearic acid: trade name “Isostearic Acid” (manufactured by Nissan Chemical Industries, Ltd.).
Stearic acid: “Refined Stearic Acid 700V” (manufactured by KAO Co.).
Palmitic acid: “EDENOR C16-98AY” (manufactured by Cognis Oleochemicals Japan Ltd.).
Behenic acid: “EDENOR C22-85JPGW” (manufactured by Cognis Oleochemicals Japan Ltd.).
1,3-BG: “1,3-Butylene Glycol” (manufactured by Daicel Chemical Industries Co.).
Glycerin: “Concentrated Glycerin for Cosmetics” (manufactured by KAO Co.).

### <Evaluation Method for Water-in-Oil Type Sunscreen Cosmetics>

### [Emulsion Stability]

The water-in-oil type sunscreen cosmetics of Examples 1-7 and Comparative Examples 1-10 are filled in 50 ml glass bottles, the state of which is examined visually after resting 2 months at 40° C., and the emulsion stability is evaluated according to the evaluation criteria below.

⊚: no separation occurs.
◯: a few water droplets on the glass bottle wall occur.
Δ: substantial water droplets on the glass bottle wall occur.
X: the cosmetic divides into two layers.

### [Spreading, Non-Oiliness, and Whitening]

Appropriate amount of cosmetics are applied on 10 exclusive panels for cosmetic evaluation, and items like “spreading”, “non-oiliness” and “whitening” are rated based on each determination criteria described below. The scores from each panel are averaged, while each item is determined based on this average score and the determination criteria below in four levels respectively.

### [Evaluation Criteria]

Score: surface state
4: very good
3: good
2: moderate
1: poor
0: very poor

### [Determination Criteria]

⊚: average score is 3.5 or more.
◯: average score is 3.0 or more but below 3.5.
Δ: average score is 2.5 or more but below 3.0.
X: average score is below 2.5.

As seen from the results shown in Table 1, all of the average scores of items including “emulsion stability”, “spreading”, “non-oiliness”, and “whitening” for water-in-oil type sunscreen cosmetics in Examples 1-7 are 3.0 or more.

Further examples of the water-in-oil type sunscreen cosmetic of the present invention are given below. In addition, the following substances are used for the components shown in Examples 8-10.

Methyl trimethicone: “TMF-1.5” (manufactured by Shin-Etsu Chemical Co.).
Decamethyl cyclopentasiloxane: “TSF-405” (manufactured by Momentive Performance Materials Japan).
Phenyl trimethicone: “KF-56A” (manufactured by Shin-Etsu Chemical Co.).
Particulate titanium dioxide: “TTO-V-4” (manufactured by Ishihara sangyo Kaisha, treated with aluminum hydroxide and stearic acid, average primary particle size 10 nm).
Particulate zinc oxide: “FINEX-50S-LP2” (manufactured by Sakai Chemical Co., treated with (dimethicone/methicone) copolymer, average primary particle size 20 nm).
Diisostearyl malate: “COSMOL 222(s)” (manufactured by the Nisshin Oillio Group Co.).
Trioctanoin: “MYRITOL GTEH” (manufactured by Cognis Oleochemicals Japan Ltd.).
Silicone branched type polyglycerin-modified silicone*²: polyglyceryl-3 polydimethylsiloxyethyl dimethicone, trade name “KF6104” (manufactured by Shin-Etsu Chemical Co.) (HLB: 4.5).
Silicone branched type alkyl•polyglycerin-modified silicone*³: laurylpolyglycerin-3 polydimethylsiloxyethyl dimethicone, trade name “KF6105” (manufactured by Shin-Etsu Chemical Co.) (HLB: 4.5).
Polyether-modified silicone*⁴: PEG10 dimethicone, trade name “KF6017” (manufactured by Shin-Etsu Chemical Co.).
Crosslinked type alkyl•polyglycerin-modified silicone*⁵: polyglyceryl-3/laurylpolydimethylsiloxyethyl dimethicone crosslinked polymer 30%+cyclopentasiloxane 70%, trade name “KSG850Z” (manufactured by Shin-Etsu Chemical Co.).
Isostearic acid: trade name “Isostearic Acid” (manufactured by Nissan Chemical Industries, Ltd.).
Polymethyl methacrylate: “Matsumoto Microsphere-M-100” (manufactured by Matsumoto Yushi-Seiyaku Co.).
KSG-16: crosslinked methylpolysiloxane 25%+dimethicone 75% (manufactured by Shin-Etsu Chemical Co., trade name).
KP-545: (alkyl acrylate/dimethicone) copolymer 30%+cyclopentasiloxane 70% (manufactured by Shin-Etsu Chemical Co., trade name).
KSP-101: (vinyl dimethicone/methicone silsesquioxane) crosslinked polymer (manufactured by Shin-Etsu Chemical Co., trade name).
Octyl methoxy cinnamate: “NOMCORT TAB-R” (manufactured by the Nisshin Oillio Group Co.).
Methyl paraben: “Methyl Paraben” (manufactured by Midori Kagaku Co., Ltd.).
Talc: “Talc JA-13R” (manufactured by Asada Milling Co.).
Mica: “EXCEL MICA JP-2” (manufactured by Miyoshi Kasei, Inc.).
1,3-BG: “1,3-Butylene Glycol” (manufactured by Daicel Chemical Industries Co.).
Glycerin: “Concentrated Glycerin for Cosmetics” (manufactured by KAO Co.).
DPG: “DPG-RF” (manufactured by ADEKA Co.).

### Example 8

### Sunscreen Cream

| | (Mixing ratio |
|---|---|
| (Components) | (mass %)) |
| 1. Methyl trimethicone | 20.0 |
| 2. Particulate titanium dioxide | 10.0 |
| 3. Particulate zinc oxide | 12.0 |
| 4. Diisostearyl malate | 2.0 |
| 5. Silicone branched type polyglycerin-modified silicone*² | 3.0 |
| 6. Isostearic acid | 0.5 |
| 7. Polymethyl methacrylate | 5.0 |
| 8. KSG-16 | 9.0 |
| 9. KP-545 | 7.0 |
| 10. Water | 25.3 |
| 11. Glycerin | 5.0 |
| 12. 1,3-BG | 1.0 |
| 13. Methyl paraben | 0.2 |

### <Preparation Method>

(1) Components 1-3 are evenly mixed with three-roll mill to give mixture A.
(2) On the other hand, components 4-9 are evenly mixed using homomixer to give mixture B.
(3) The mixture A and mixture B obtained above are evenly mixed by homomixer to yield oil phase mixture C.
(4) On the other hand, component 13 is dissolved in component 12 by heating, in which components 10 and 11 are added, followed by mixing with homomixer to yield aqueous phase mixture D.
(5). The above resulting aqueous phase mixture D is added into the oil phase mixture C obtained above and mixed by homomixer so as to emulsify followed by degassing, and then filled in containers to give water-in-oil type sunscreen cream.

### <Evaluation>

The obtained sunscreen cream is evaluated as above, and the results are: emulsion stability: “⊚”, spreading: “◯”, non-oiliness: “⊚”, whitening: “⊚”.

### Example 9

### Cosmetic Base

| | (Mixing ratio |
|---|---|
| (Components) | (mass %)) |
| 1. Decamethyl cyclopentasiloxane | 23.0 |
| 2. Phenyl trimethicone | 4.0 |
| 3. Particulate titanium dioxide | 2.0 |
| 4. Particulate zinc oxide | 10.0 |
| 5. Trioctanoin | 7.0 |
| 6. Silicone branched type polyglycerin-modified silicone*³ | 2.0 |
| 7. Polyether-modified silicone*⁴ | 1.5 |
| 8. Isostearic acid | 0.5 |
| 9. Polymethyl methacrylate | 6.0 |
| 10. KSG-16 | 6.0 |
| 11. Octyl methoxy cinnamate | 6.0 |
| 12. Talc | 1.0 |
| 13. Mica | 1.0 |
| 14. Water | 21.8 |
| 15. Glycerin | 2.0 |
| 16. DPG | 4.0 |
| 17. 1,3-BG | 2.0 |
| 18. Methyl paraben | 0.2 |

### <Preparation Method>

(1) Components 2-5 are evenly mixed with three-roll mill to give mixture A.
(2) On the other hand, components 1 and 6-13 are evenly mixed using homomixer to give mixture B.
(3) The mixture A and mixture B obtained above are evenly mixed by homomixer to yield oil phase mixture C.
(4) On the other hand, component 18 is dissolved in component 17 by heating, in which components 14-16 are added, followed by mixing with homomixer to yield aqueous phase mixture D.
(5). The above resulting aqueous phase mixture D is added into the oil phase mixture C obtained above and mixed by homomixer so as to emulsify followed by degassing, and then filled in containers to give water-in-oil cosmetic base.

### <Evaluation>

The obtained cosmetic base is evaluated as above, and the results are: emulsion stability: “⊚”, spreading: “◯”, non-oiliness: “◯”, whitening: “⊚”.

### Example 10

### Cosmetic Base

| | (Mixing ratio |
|---|---|
| (Components) | (mass %)) |
| 1. Decamethyl cyclopentasiloxane | 27.8 |
| 2. Particulate zinc oxide | 18 |
| 3. Silicone branched type polyglycerin-modified silicone*² | 3.5 |
| 4. Isostearic acid | 0.5 |
| 5. KSP-101 | 3 |
| 6. Crosslinked type alkyl•polyglycerin-modified silicone*⁵ | 2 |
| 7. Talc | 1 |
| 8. Mica | 3 |
| 9. Water | 37 |
| 10. DPG | 2 |
| 11. 1,3-BG | 2 |
| 12. Methyl paraben | 0.2 |

### <Preparation Method>

(1) Components 2-5 are evenly mixed with three-roll mill to give mixture A.
(2) On the other hand, components 1 and 6-8 are evenly mixed using homomixer to give mixture B.
(3) The mixture A and mixture B obtained above are evenly mixed by homomixer to yield oil phase mixture C.
(4) On the other hand, component 12 is dissolved in component 11 by heating, in which components 9 and 10 are added, followed by mixing with homomixer to yield aqueous phase mixture D.
(5). The above resulting aqueous phase mixture D is added into the oil phase mixture C obtained above and mixed by homomixer so as to emulsify followed by degassing, and then filled in containers to give water-in-oil cosmetic base.

### <Evaluation>

The obtained cosmetic base is evaluated as above, and the results are: emulsion stability: “⊚”, spreading: “⊚”, non-oiliness: “⊚”, whitening: “⊚”.

What is claimed is:

1. A water-in-oil type sunscreen cosmetic, which comprises particulate zinc oxide subjected to surface hydrophobizing treatment and/or particulate titanium dioxide subjected to surface hydrophobizing treatment; silicone branched type polyglycerin-modified silicone; and isostearic acid, wherein
the content of the silicone branched type polyglycerin-modified silicone is 1.5-10% by mass on the basis of the total amount of the cosmetic, and
the content of the isostearic acid is 0.1-10% by mass on the basis of the total amount of the cosmetic.

2. The water-in-oil type sunscreen cosmetic according to claim 1, wherein the surface of the particulate zinc oxide and the particulate titanium dioxide is subjected to hydrophobizing treatment with silicone oil or metallic soap.

3. The water-in-oil type sunscreen cosmetic according to claim 1 or 2, wherein the average primary particle size of the particulate zinc oxide and the particulate titanium dioxide is 1-50 nm.

4. The water-in-oil type sunscreen cosmetic according to any one of claims 1 to 3, wherein the HLB value of the silicone branched type polyglycerin-modified silicone is 2-5.

5. The water-in-oil type sunscreen cosmetic according to any one of claims 1 to 4, wherein the cosmetic does not contain any organic UV absorbent.
